(19) United States
(12) Patent Application Publication
(10) Pub. No.: US 2005/0032197 A1
(43) Pub. Date: Feb. 10, 2005

(54) Membrane protein interaction assays

(76) Inventors: Marc F. Pelletier, Branford, CT (US); Thomas David Y., Montreal (CA)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/494,787

(22) PCT Filed: Nov. 6, 2002
(86) PCT No.: PCT/CA02/01718

Related U.S. Application Data
(60) Provisional application No. 60/331,012, filed on Nov. 6, 2001.

(30) Foreign Application Priority Data
Nov. 6, 2001 (CA) 2,361,118

(51) Int. Cl. C12N 1/18; C12Q 1/00

(52) U.S. Cl. 435/254.2; 435/4

(57) ABSTRACT

In some aspects, the invention provides systems for analysis of protein-protein interactions in which the luminal domain of Ire1p, or homologues thereof, is replaced with the domains of heterologous proteins of interest that then mediate either homo- or heterodimerization of the chimeric fusion proteins to activate a reporter system that may be based on the unfolded protein response pathway (similar to the native activity of Ire1p).

### FIELD OF THE INVENTION

The invention is in the field of molecular biology, particularly products and processes for measuring and testing molecular interactions of cell membrane bound proteins in cells, involving the use of recombinant nucleic acids.

### BACKGROUND OF THE INVENTION

With the advent of the classical two-hybrid assay proposed by Fields and Song (1989), a plethora of intracellular protein-protein interactions have been found and characterized. This genetically based system complemented the standard biochemical approaches, which include methods such as chemical cross-linking, co-immunoprecipitation, co-fractionation by chromatography, GST-pull down assays, and far-western analysis. The interactions found with the Fields and Song system, when compiled into networks, provided detailed protein interaction maps, greatly enhancing our understanding of the inherent biological significance of each protein therein.

Some of the limitations of conventional embodiments of the two-hybrid system, which is based on the re-assembly of the Gal4p transcriptional activator in vivo, through direct interactions of the fused proteins of interest, have been overcome through the development of alternative approaches. These include the split-ubiquitin system (Stagljar, Korostensky, et al. 1998; Johnsson & Varshavsky 1994), the SOS and Ras recruitment systems (SRS and RRS) (Aronheim, Zandi, et al. 1997; Broder, Katz, et al. 1998), G-protein fusions (Ehrhard K N, Jacoby J J, et al. 2000), and the oligomerization-assisted enzymatic complementation systems which result in the reassembly of murine dihydrofolate reductase (mDHFR) (Pelletier, Arndt, et al. 1999) or the β-galactosidase (Rossi, Blakely, et al. 2000) from E. coli. These techniques have broadened the spectrum of proteins analyzed, to include transcriptional activators that tend to self-activate the Gal4p based system (Aronheim, Zandi, et al. 1997), intergral membrane and membrane-associated proteins which are topologically restricted from the nucleus (Stagljar, Korostensky, et al. 1998; Broder, Katz, et al. 1998; Ehrhard K N, Jacoby J J, et al. 2000), and also permitted the study of protein-protein interactions endogenously within mammalian cells. Regardless of the inherent constraints of the classical yeast two-hybrid system, it has been selected for the formidable endeavor of comprehensively mapping the protein-protein interactions within the nematode Caenorhabditis elegans (Walhout, Boulton, et al. 2000) and yeast Saccharomyces cerevisiae (Uetz, Giot, et al. 2000; Schwikowski, Uetz, et al. 2000) for which the complete genomes are now available (Goffeau A, Barrell B G, et al. 1996).

### SUMMARY OF THE INVENTION

In some aspects, the invention provides systems for analysis of protein-protein interactions in which the luminal domain of Ire1p, or homologues thereof, is replaced with the domains of proteins of interest that then mediate either homo- or heterodimerization of the chimeric fusion proteins to activate a reporter system that may be based on the unfolded protein response pathway (similar to the native activity of Ire1 p).

### BRIEF DESCRIPTION OF THE DRAWINGS

FIG. 1. A putative model for the UPR in yeast. The accumulation of unfolded proteins in the ER leads to Ire1p oligomerization, by an unknown ligand in the ER or perhaps through the titration of the ER Hsp70 homologue Bip (Bertolotti, Zhang, et al. 2000). Oligomerization permits transphosphorylation of the kinase domains, this in turn leads to activation of the intrinsic ribonuclease activity. HAC1 mRNA is spliced and ligated, exported to the cytoplasm, and translated. The translated Hac1p protein is imported into the nucleus and combines with other transcription factors to increase the rate of transcription of ER molecular chaperones and folding enzymes.

FIG. 2. Schematic diagram of the constructs used in the membrane one- and two-hybrid systems. The vectors pMP1 (LEU2) and pMP2 (HIS3) encode the Ire1p signal sequence (SS), a multiple cloning site (MCS), the Ire1p transmembrane (TM) and the cytosolic domains Ire1p. The vectors pMP3 and pMP4 express mutant forms of the cytosolic domains of Ire1p, either Ire1p_{K702R} (Shamu & Walter 1996) (pMP3) and Ire1 p_{P982} (Shamu & Walter 1996) (pMP4). pMP1 through pMP4 are under the control a truncated IRE1 promoter (411 bp 5′ to the start codon). pMP5 expresses a highly active UPR transcription activator. It consists of a HAC1::VP16 fusion that is under the control of the Ire1p endonuclease activity. Ire1p endonuclease activity splices the HAC1 intron, placing the transcriptional domain of VP16 (aa 410-490) in frame with the DNA binding domain of Hac1p.

FIG. 3. Schematic diagram of the trans-phosphorylating membrane protein two-hybrid system. Dimerization of proteins X and Y, expressed as fusions with the Ire1p (pMP1/pMP2, or mutant proteins Ire1p_{K702R} and Ire1pP₉₈₂, pMP3/pMP4respectively) leads to the trans-phosphorylation of the Ire1p kinase domain. This will subsequently activate the endoribonuclease domain of Ire1p, which processes the mRNA of the transcriptional activator Hac1p. Hac1p will then induce the expression of the reporter genes. In the forward approach to protein-protein analysis, the strains used are yMP1 and yMP2. Upon dimerization of the Ire1p fusion proteins in these strains, the UPR-Y::LacZ and UPR-Y::ADE2 reporters are induced, and can the cells can be assayed by growth on synthetic dropout medium (SD) lacking inositol, for β-galactosidase activity, or by the white phenotype resulting from the expression of the ADE2 gene. Strains yMP3 and yMP4 are used for the first reverse approach to membrane two-hybrid protein-protein analysis. In this case, if Ire1p fusion proteins are expressed that lead to dimerization, the UPR-Y::CAN1, UPR-Y::URA3, and UPR-Y::ADE2 reporters will be induced. Expression of CAN1 and URA3 inhibits growth in the presence of canavanine and 5-FOA, respectively. Expression of ADE2 should lead to white colonies. Putative inhibitors of the membrane protein-protein interactions can be assessed by monitoring growth of the reporter strains in the presence of these compounds, respectively, and through the expression of a red phenotype. The second approach to reverse membrane protein two-hybrid analysis uses the strains yMP5 and yMP6. These strains have two integrated reporters, UPR-Y::LexA::TUP1 and LexA-OP::ADE2. If Ire1p fusions interact, the first reporter, UPR-Y::LexA::TUP₁, will be induced. The encoded protein (LexA::TUP1p) will then bind the LexA operator (LexA-OP) of the second reporter blocking the expression of the ADE2 gene product. If the interaction of the membrane fusion proteins is inhibited, the cells will be able to grow in the absence of adenine (SD-adenine).

FIG. 4. Interactions between calnexin and calreticulin, with ERp57, using the Ire1p based two-hybrid system, and functional complementation of the lumenal domain of Ire1p, by fusions with Ire1p that lead to mono/heterodimerization. (A) Schematic diagram of the constructs used to test the ER protein two-hybrid system are shown. The parental vectors pLJ89 (LEU2) and pLJ96 (HIS3) encode the Ire1p signal sequence (SS), a 23 aa linker in frame with the Ire1p transmembrane (TM) and kinase/endoribonuclease domains, under the control the IRE1 promoter. Wild-type IRE1 was also subcloned into the plasmid that was parental to pLJ89 (Wt). Clone accession numbers are indicated in parenthesis. (B) A beta-galactosidase filter assay was performed on S. cerevisiae diploid strains expressing Ire1p fusion proteins. Yeast cell patches prior to transfer are shown on right. Crosses were made with strains carrying pLJ89 and pLJ96 as negative controls, while the extracellular domain of the murine erythropoietin receptor (EPOr) served as a positive control. (C) Quantitative permeable cell/beta-galactosidase assays were performed on yeast cells in the absence or presence of 5 micro g/ml of tunicamycin for 1 h. beta-galactosidase activity is reported in the absence of tunicamycin (lightly shaded bars) and for treated cells. (dark bars). Plasmid combinations are indicated, and the parental W303a strain (with the integrated UPR reporter) was used as a positive control (Wt). beta-galactosidase units are defined as [A₄₂₀×1000]/[A₆₀₀ of cells×culture vol. (ml)×reaction time (min)], and error bars represent standard deviation, n=3.

FIG. 5. The loop domains of calnexin and calreticulin are suffient, and the B thioredoxin domain of ERp57 is required for the heterodimerization of both calnexin and calreticulin, with ERp57. (A) Schematic diagram of the constructs used to map the protein-protein interaction domains of calnexin (CNX), calreticulin (CRT), and ERp57. The loop domains of CNX and CRT, with their corresponding type 1 and type 2 repeat configuration, and the thioredoxin domains of ERp57 (A, B, B′, A′) are shown. (B) beta galactosidase filter assays on strains expressing the mapping fusion proteins described in A are shown. pLJ89/pLJ96 and EPOr were used respectively as negative and positive controls.

FIG. 6. The loop domain of calnexin interacts directly with ERp57 in vitro. (A) GST fusions consisting of the full length lumenal domain of CNX (CNX_{K46-M417}, lane 1), the loop domain (or P-region, CNX_{M267-L412}, lane 2), and GST alone (lane 3) were purified and are shown on a 10% SDS-PAGE. (B) The purified fusion proteins were loaded onto columns with Glutathione Sepharose 4B, followed by purified ERp57³. The columns were washed, the proteins were then eluted with reduced glutathione, and a Western blots performed on the eluant with anti-ERp57 antiserum.

### DETAILED DESCRIPTION OF THE INVENTION

In some aspects, the invention provides systems for analysis of protein-protein interactions in which the luminal domain of Ire1p, or homologues thereof, is replaced with the domains of proteins of interest that then mediate either homo- or heterodimerization of the chimeric fusion proteins to activate a reporter system that may be based on the unfolded protein response pathway (similar to the native activity of Ire1p).

In one aspect, the invention provides a ‘forward’ approach to membrane protein-protein interactions analysis. In one such embodiment, the invention provides an Ire1p-based membrane protein ‘one-hybrid’ (i.e. homodimer-activated) system. In alternative embodiments, the invention provides ‘two-hybrid’ (i.e. heterodimer-activated) systems. Both the membrane protein one- and two-hybrid systems may use adaptations of the unfolded protein response (UPR) in Saccharomyces cerevisiae to report hetero or homodimerization of Ire1p-derived fusion proteins. In such systems of the invention, protein-protein interactions for both soluble and membrane proteins may be detected upon dimerization of fusion proteins comprising a truncated form of Ire1p.

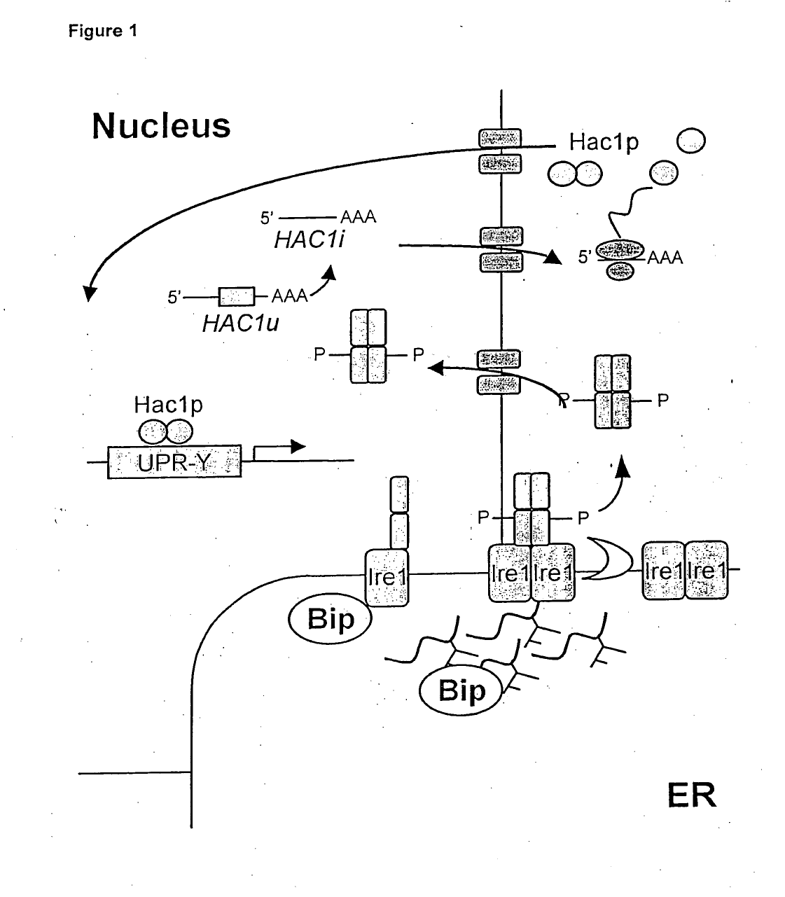

In some embodiments, the genes of interest are subcloned in the place of the lumenal domain of Ire1p (an ER transmembrane kinase/endoribonuclease), which is known to mediate its dimerization upon the accumulation of unfolded proteins in the ER (Shamu & Walter 1996) (FIG. 1). Oligomerization leads to the transphosphorylation of the kinase domains, and in turn the activation of its endoribonuclease domain (Shamu & Walter 1996). This endoribonuclease domain, then targeted to the nucleus, processes HAC1 mRNA (Sidrauski & Walter 1997). The putative mechanism of some systems of the invention involves processing of HAC1 mRNA to remove the translational attenuation permitting the translation of the bZIP transcriptional activator, which then binds the yeast UPR element (UPR-Y).

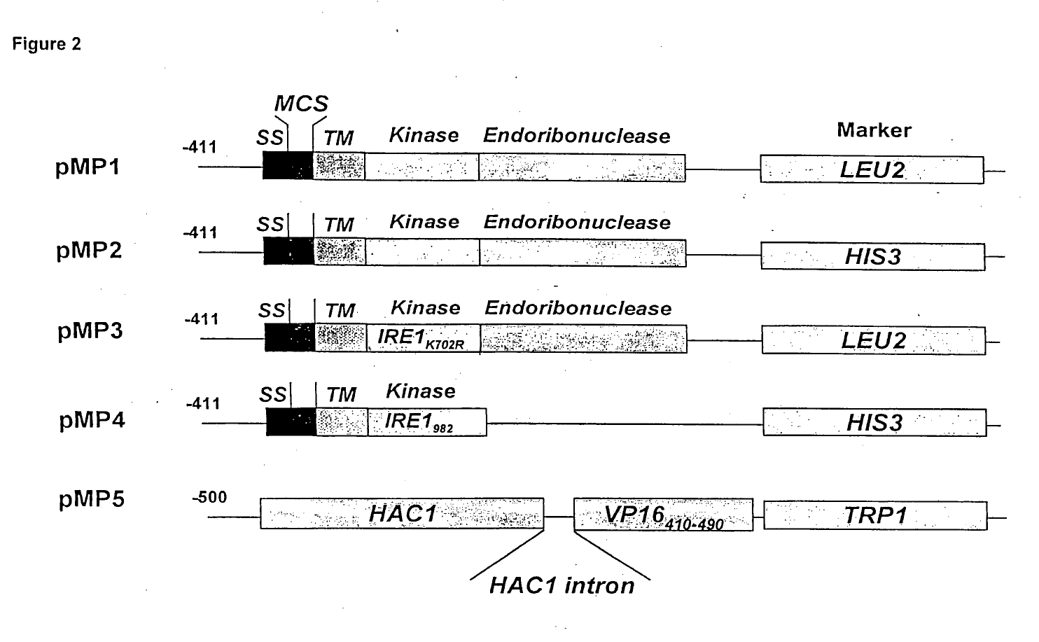

The parental plasmids pMP1 and pMP2 (FIG. 2) encode (1) 411 bp of the IRE15′ untranslated promoter region, (2) the Ire1p signal sequence, (3) a 69 bp multiple cloning site, and (4) the transmembrane domain (TM) and cytosolic domains of Ire1p. pMP1 and pMP2 carry the yeast metabolic markers LEU2 and HIS3 respectively.

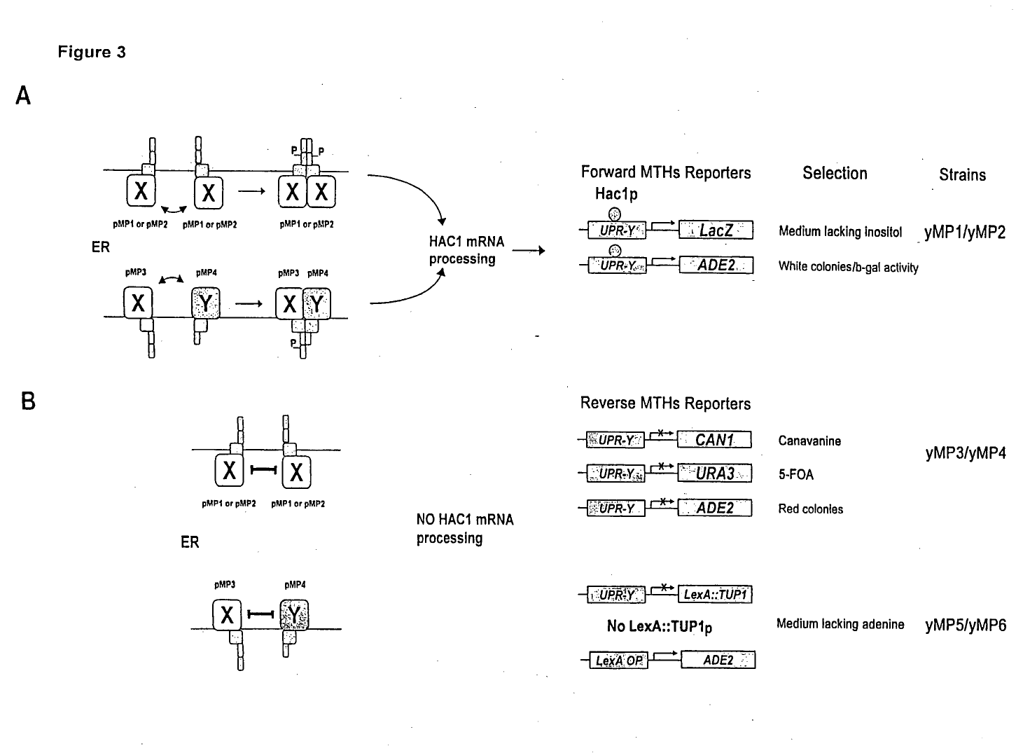

The reporter strains for the forward membrane one- and two-hybrid systems, strains yMP1 and yMP2 (Mata ΔIRE1 ade2-1 UPR-Y::CYC1::ADE2 UPR-Y::CYC1::LacZ and Mat alpha ΔIRE1 ade2-1 UPR-Y::CYC1::ADE2 UPR-Y::CYC1::LacZ, respectively), have an integrated LacZ gene under the control of a UPR-Y fused to a truncated CYC1 promoter to report fusion protein heterodimerization. The genes of interest (X and Y) are amplified by high fidelity PCR, and subcloned directly into either pMP1 or pMP2 plasmids (or pMP3 and pMP4) between the regions encoding the linker and the TM domain (or 3′ of the region encoding the TM, if the endogenous TM is used) by recombinational cloning in the reporter yeast strains. Protein-protein interactions lead to the induction of the UPR-Y::CYC1::LacZ reporter (FIG. 3A), and are detected by (1) liquid beta-galactosidase assay, (2) permeabilized-cell filter assay for beta-galactosidase, (3) by monitoring growth in synthetic dropout medium (SD) without supplemental inositol, or (4) selection of white colonies on adenine rich medium. ΔIRE1 strains grow slowly in the absence of inositol, unless there is functional complementation by the heterodimerizing fusion proteins.

In some embodiments, particularly Ire1p-based membrane two-hybrid systems, to help optimize a process in which the fusion proteins only or predominantly activate the UPR pathway in trans (a characteristic of two-hybrid systems), mutant kinase domains, such as those that are described by Shamu and Walter, 1996, may be used. For example, the mutant Ire1p kinase domain encoded in pMP3 (FIG. 2) has a single point mutation in the kinase active site (K702R). This mutation reportedly reduces the ability of this form Ire1p to activate UPR alone by 75% (Shamu & Walter 1996). Similarly, the kinase domain in pMP4 (FIG. 2) encodes a mutation resulting in a stop codon at amino acid residue 983 of Ire1p. This mutation leads to the deletion of the endoribonuclease domain, and cannot activate the UPR alone (Shamu & Walter 1996). In some embodiments, the same reporter strains can be used for these plasmids as for pMP1 and pMP2.

In alternative embodiments, the invention provides a ‘reverse’ approach to soluble/membrane protein-protein interaction analysis. For example, a reverse membrane protein two-hybrid system may use alternative methods to report the interruption of soluble or membrane protein-protein interactions detected with the Ire1p based system. A first example of such an embodiment is based on the expression of CAN1, URA3, and ADE2 under the control of UPR-Y::CYC1, while a second exemplary embodiment integrates the technology described above (forward approach) with the repressed trans-activator (RTA) system described in the U.S. Pat. No. 5,885,779 issued to Sadowski, et al.

In one aspect, the invention provides a reverse membrane protein two-hybrid system using UPR-Y::CYC1::CAN1, UPR-Y::CYC1::URA3, and UPR-Y::CYC1::ADE2 reporters. The yeast strains yMP3 and yMP4 (Mata ΔIRE1 ade2-1 UPR-Y::CYC1::ADE2 UPR-Y::CYC1::CAN1 UPR-Y::CYC1::URA3 and Mat alpha ΔIRE1 ade2-1 UPR-Y::CYC1::ADE2 UPR-Y::CYC1::CAN1 UPR-Y::CYC1::URA3, respectively) have an integrated CAN1 open reading frame under the control of the UPR-Y reporter. If proteins that heterodimerize are expressed as Ire1p fusions in pMP3 and pMP4, in the strains yMP3 or yMP4, CAN1 expression will be induced (FIG. 3B). Under these conditions the strains will be unable to grow in the presence of canavanine, which is a toxic analogue to arginine{Hoffmann #8150}. Similarly, dimerization also leads to the upregulation of the URA3 reporter (FIG. 3B). In the presence of the compound 5-fluoro-orotic acid (5-FOA), cells expressing high levels of URA3 exhibit growth inhibition (Boeke et al. 1984). Interruption of the protein-protein interactions between Ire1p fusions, when using either the CAN1/canavanine or URA3/5-FOA reporter systems will lead to normal cell growth.

When the strains yMP1 through yMP4, which all have the intergrated UPR-Y::CYC1::ADE2 reporter, are (1) transformed with constructs expressing Ire1p fusion proteins that homo- or heterodimerize (pMP1 or pMP2, or pMP3 and pMP4, repectively), and (2) the protein-protein interaction is blocked, yeast colonies will exhibit a red phenotype in these strains. In some embodiments these strains must be plated on medium that is enriched with adenine to observe the phenotype.

In some embodiments, the invention provides a reverse membrane protein two-hybrid with the repressed trans-activator (RTA) reporter system. For example, this approach may consist of monitoring the interruption of protein-protein interactions, by blocking the expression of a LexA::TUP1p fusion protein (LexA DNA binding domain::TUP1₁₋₂₀₀). If proteins that heterodimerize are expressed as Ire1p fusions in pMP3 and pMP4, in the yeast strains yMP5 and yMP6 (Mata ΔIRE1 ade2-1 UPR-Y::CYC1::LexA:::TUP1 LexA-oper::ADE2 and Mat alpha ΔIRE1 ade2-1 UPR-Y::CYC1::LexA::TUP1 LexA-oper::ADE2, respectively), the fusion protein LexA::TUP1p will be induced. Expression of LexA::TUP1p will lead to the repression of the reporter ADE2-OP::LexA::ADE2 (FIG. 3B). Thus, cell growth will occur when the strains are grown in the presence of an inhibitor of the interaction.

In some embodiments, the invention may provide enhanced reporter activation by Hac1::VP16p transcriptional activator fusion. For example the activity of the above reporters for the MTHs may be enhanced by modifying the UPR transcriptional activator Hac1p. The modification consists of removing the region of the gene that encodes the last 18 amino acids (aa) of HAC1, and replacing it with the sequence coding for the acidic region of the strong transcriptional activator VP16 (aa 410-490) (FIG. 2). Unless the UPR is activated through the dimerization of the Ire1p fusion proteins encoded on pMP3 and pMP4, the HAC1::VP16 mRNA will not be spliced by the Ire1p endoribonuclease encoded on pMP1. In the absence of this mRNA splicing, a stop codon in the HAC1 intron terminates translation before reaching the region encoding the VP16 activation domain. Thus mRNA splicing, brought on by the dimerization of the Ire1p fusion proteins of the MTHs, is a required event to bring the region encoding the VP16 activation domain into the coding frame.

The plasmid required for the enhanced reporter activity consists of the HAC1::VP16 gene fusion placed under the control of the HAC1 promoter (500 bp of untranslated sequence 5′ of the HAC1 ORF), and includes a TRP marker. This plasmid (pMP5) will lead to a greater activation of the reporters when transformed into any of the above strains, and maintained on the appropriate synthetic droupout medium without tryptophan.

In use, in some embodiments, the invention may facilitate analysis of organelle specific protein-protein interactions. For example, the invention may provide protein interaction maps of the human endoplasmic reticulum including membrane and lumenal proteins. In such embodimets, a database of genes encoding proteins of the human ER may be provided. Proteins may for example be designated as ER localized on the basis of experimental results and using the PSORT application found online at http://psort.nibb.ac.jp. Genes encoding these ER proteins can be amplified by PCR from or from full length cDNA libraries. Such genes may be sublconed into the MTHs (pMP1 and pMP2, first linearized with SalI) through recombination cloning (Uetz, Giot, et al. 2000). Briefly, 5′ and 3′ primers are designed to include 15-18 bases that are specific to the 5′ and 3′ ends of gene to be amplified, respectively, and include 35 bases that are identical to the pMP1 or pMP2 at the Sal1 site, for homologous recombination. 1 ug of PCR product is included in a S. cerevisiae tranformation mix (50 ul of cells, 0.1 M LiAc, 5 ug salmon sperm DNA, 300 ul of polyethylene glycol (M.W. 3300) with 0.5 ug of linearized pMP3 or pMP4. Transormants are selected on the appropriate synthetic dropout medium including myo-inositol. Complete libraries of clones can be made in both plasmids, and introducted into strains of opposite mating types (yMP1 and yMP2). These can then be crossed in a comprehensive matrix approach, and assayed for protein-protein interactions using a forward membrane two-hybrid system of the invention.

In some embodiments, the systems of the invention may also provide a means for the analysis of the protein-protein interactions in a variety of organelles, such as the Golgi, nucleus and the lumen of the mitochondial matrix. For example, to adapt the system of the invention to analyze proteins from organelles other that the ER, the region of pMP3 and pMP4 encoding the IRE1 ER signal sequence may be removed. Clones can be selected (1) in a process similar the one used in the creation of the human ER cDNA library (described above), (2) amplified on the basis of experimental results demonstrating their localization, or (3) based on the results from PSORT queries (URL).

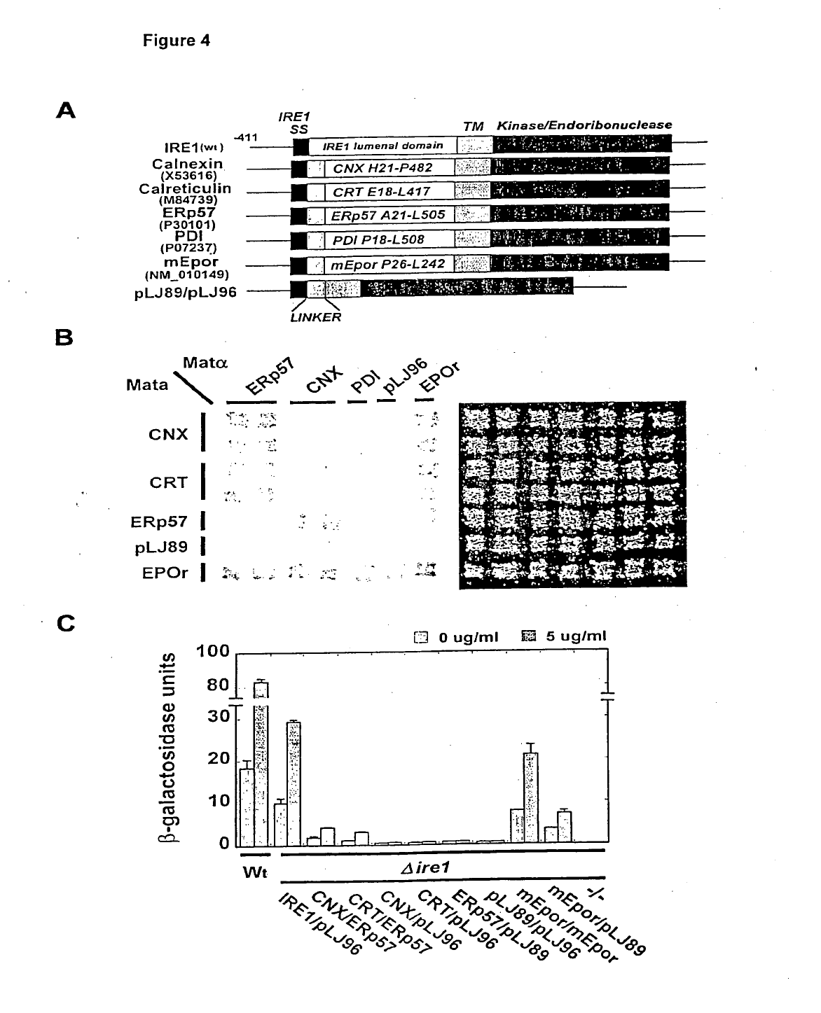

In an examplary embodiment (FIGS. 4, 5 and 6) the system of the invention has been used to map the regions of protein-protein interaction of calnexin and its soluble homologue calreticulin with ERp57, and to show that protein disulfide isomerase (PDI) does not interact. The system was used to define domains of calnexin/calreticulin and ERp57 that interact and the interaction of these fragments was verified by their physical association in vitro.

For the embodiment used to collect the data shown in FIGS. 4, 5 and 6, we first integrated a cassette with the LacZ gene from E. coli under the control of a chimeric promoter into Saccharomyces cerevisiae (W303a). This promoter consists of a minimal yeast UPRE fused to a truncated CYC1 promoter. The reporter strain was then created by crossing this strain with BY4742 (Δire1), and haploids were isolated that carried both the integrated reporter with the Airel genotype. This strain cannot respond with a UPR to agents that induce this in the wild-type (results not shown).

The plasmids pLJ89 and pLJ96 (FIG. 4A), with LEU2 and HIS3 markers respectively, encode Ire1p with the N-terminal lumenal domain deleted up to the transmembrane domain (TM). The Ire1p signal sequence is intact and followed by a 23 amino acid linker to ensure that the signal peptidase does not cleave the nascent protein subcloned in place of the Ire1p ER lumenal domain. The genes to be inserted were amplified by high fidelity PCR using primers with plasmid homology regions, and subcloned directly into yeast by recombinational cloning between the homologous regions of the linker and the TM domains. The endogenous promoter of IRE1 was subcloned and used to express the fusion proteins since overexpression has been shown, at least for the mammalian homologue, to induce UPR²³.

To test our approach, MATa strains expressing a first set of Ire1p fusions were crossed with MATA strains expressing a second set of fusions (FIG. 4B). The first set consisted of fusions containing the lumenal domain of CNX, complete CRT, complete ERp57, the parental vector pLJ89 (negative control), and the extracellular domain of the murine erythropoietin receptor (EPOr), as a positive control, respectively (FIG. 4B). The second set of fusions were ERp57, the lumenal domain of CNX, the ER protein disulfide isomerase (PDI), the parental vector pLJ96 (negative control), and again the extracellular domain the EPOr (positive control), respectively (FIG. 4B). Diploid strains expressing both sets of fusion proteins were isolated, transferred to nitrocellulose, and tested for β-galactosidase activity (FIG. 4B).

It is thought that ERp57 functionally interacts with CNX and CRT to specifically promote folding of bound glycoproteins. The homologous ER protein PDI does not functionally interact in this way. Also, it has been shown that ERp57 but not PDI can be cross-linked to CNX and CRT, implying a physical as well as a functional interaction. With these proteins in the ER protein two-hybrid system of the invention, we show that this specificity is maintained. That is we detect the interaction of CNX and CRT with ERp57 and not with PDI (FIG. 4B). Also, when CNX and ERp57 were subcloned into the plasmids pLJ89 and pLJ96 in a reciprocal combination, the interaction was still observed (FIG. 4B). From these results, we also conclude that CNX does not interact with CRT, nor does ERp57 interact with PDI. In addition, we did not find homodimerization for any of these fusion proteins (FIG. 4B). We used the extracellular domain of the murine erythropoietin receptor (EPOr) as a positive control, as it does mediate homodimerization of this receptor in mammalian cells. It has been shown for the EPOr that the extracellular domains are already dimerized and that ligand binding causes a change in the orientation of the cytosolic JAK-2 kinase domains, which then become activated through a trans-phosporylation event²⁴⁻²⁶. Thus this interaction is seen in the ER protein two-hybrid system as LacZ positives in the diploid cells (FIGS. 4B and C) and also in haploid cells (results not shown).

A ligand-independent activation model for Ire1p was recently proposed by Liu et al.,²⁷, who found that a leucine zipper homodimerization domain could replace the lumenal domain of Ire1p, and the fusion protein could also sense the accumulation of unfolded proteins within the ER. We tested whether this was also true for heterodimerizing fusions if Ire1p. We performed quantitative galactosidase assays on cells from the previous experiment (FIG. 4C). To induce the UPR, cells were grown in the presence of 5 g/ml of the glycosylation inhibitor tunicamycin. In both cases, either homo- and heterodimeriztion, the UPR was activated, when compared to the control strains (FIG. 4C). This supports the finding that the response to unfolded proteins does not specifically require the Ire1p lumenal domain²⁷. It also suggests that fusions that lead to both homo- and heterodimerization will functionally complement Ire1p, and also that our fusions are correctly localized.

Interestingly, a dose dependent response was seen for the diploid strains (and haploid strains, results not shown) expressing the murine EPOr, with a level of response similar to the strain expressing Ire1p (FIG. 4C). However, the tunicamycin induced UPR in the strain with the plasmid borne Ire1p is weaker than found in the wild-type W303a strain (FIG. 4C). To account for this difference, it is possible that 411 bp 5′ of the ATG codon of IRE1 is an incomplete promoter, insufficient for the expression of a full complement of IRE1. To confirm that these parental plasmids target the fusion proteins correctly to the ER, we subcloned GFP as the lumenal domain of Ire1p and placed this fusion under the control of much stronger GAL1 promoter, and even at high expression levels the fusion protein displayed a perinuclear localization typical of ER membrane proteins in S. cerevisiae (data not shown).

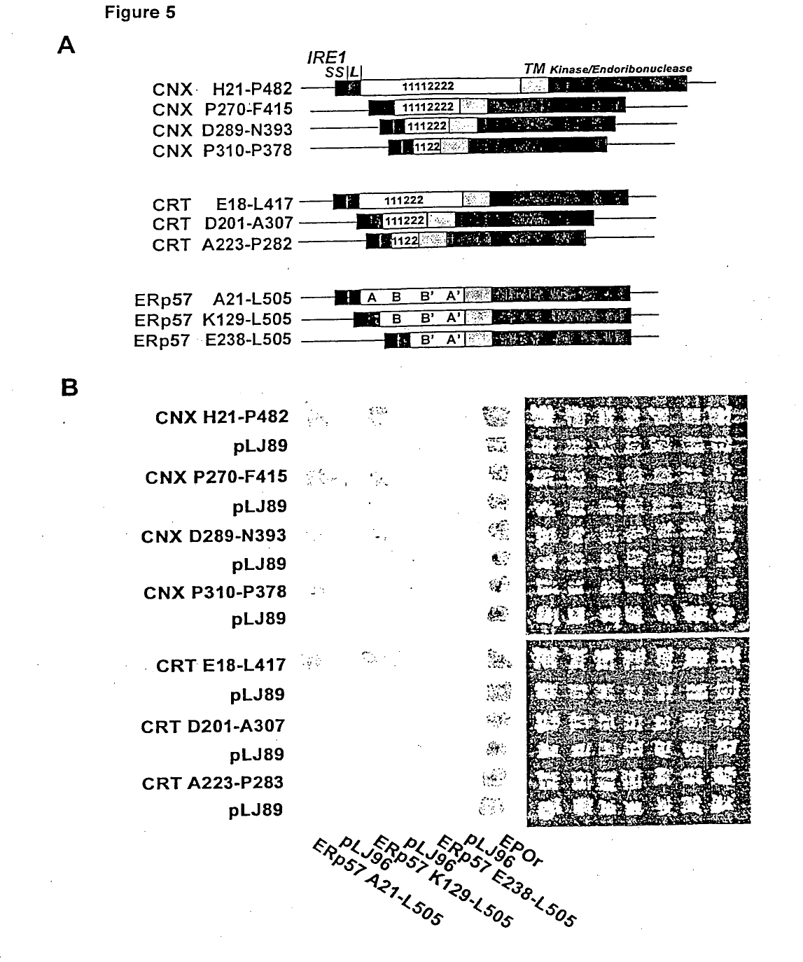

Next, we mapped the protein interactions between CNX and CRT, with ERp57. When tested in our two-hybrid system, the CNX lectin domain failed to mediate a specific interaction with ERp57 (data not shown), while its loop domain (P₂₇₀-F₄₁₅) did confer this specificity. To map the region of interaction, we therefore designed constructs of the loop domains of CNX and CRT (FIG. 5A). To summarize, in addition to the full lumenal domains of CNX and CRT, we made three additional CNX, and two additional CRT constructs. We subcloned (1) the region encoding the tip of the CNX loop, which contains two of each repeat motif (repeats 1122, P₃₁₀-P₃₇₈), (2) a longer loop with three of each repeat (111222, D₂₈₉-N₃₉₃), (3) and one which has four of each and forms the entire loop domain (11112222, P₂₇₀-F₄₁₅) (FIG. 5A). Similarly for CRT, we subcloned the tip region (1122, A₂₂₃-P₂₈₃), and the full CRT loop (111222, D₂₀₁-A₃₀₇) (FIG. 5A).

The constructs for mapping ERp57 were based on the proposed domain structure of its sequence-related homologue PDI³⁰. Both PDI and ERp57 contain four thioredoxin domains in tandem (A-B-B′-A′). The A and A′share high similarity to thioredoxin, and each contain a copy of the active site consensus sequence —C-G-H—C—, with the N-teminal cysteine being reactive, while the B and B′ domains, which are less conserved, have lost this active site consensus sequence³⁰. In addition to the full-length ERp57 fusion construct, we made two deletion constructs. The first had the first thioredoxin domain removed (leaving B-B′-A′, K₁₂₉-L₅₀₅), and the second had two removed (leaving B′-A′, E₂₃₈-L₅₀₅) (FIG. 5A). To test for protein interactions, the CNX and CRT constructs were transformed into the MATa reporter strain, while the ERp57 constructs, into the MAT reporter strain. The haploid strains were streaked, the diploids selected, and the interactions were verified with the p-galactosidase filter assay (see FIG. 5B). The parental plasmids pLJ89 and pLJ96, and the extracellular domain of EPOr were used as negative and positive controls, respectively. The results of this experiment show that two sets of repeat motifs, hence the tip of loop domain (with a “1122” repeat configuration) is sufficient to mediate the interaction of CNX and of CRT, with ERp57 (FIG. 5B). Moreover, the results show that the second thioredoxin domain of ERp57 (B) is required to interact with both CNX and CRT (FIG. 5B). This result further confirms the specificity of our system, and also demonstrates its sensitivity for detecting the interaction of small domains (60 aa in the case of CRT). In addition to the functional assay³ and the cross-linking results², our results show that there are specific interactions between the regions of CNX and CRT, and ERp57, that we have mapped. While ERp57 and PDI are homologous (33% identity), the conserved regions are concentrated in the A and A′ thioredoxin domains, which each contain an active-site sequence. The B and B′ domains of ERp57 and PDI show a low degree of similarity³⁰, and explain the specificity that we detected here.

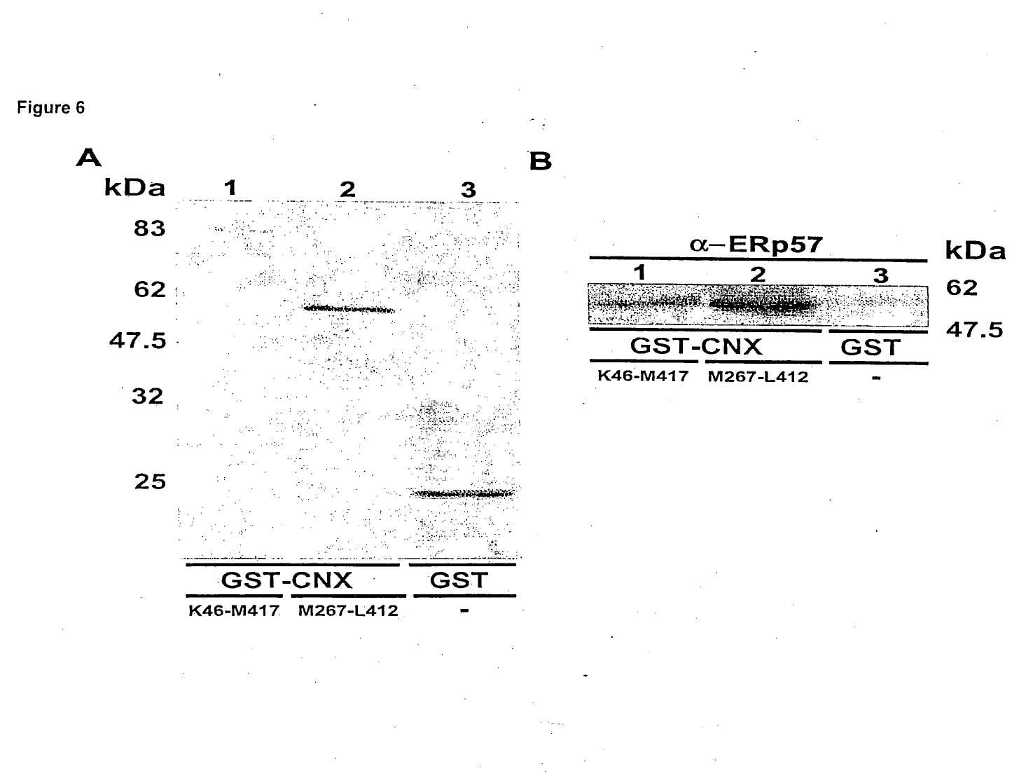

To confirm that these two-hybrid results can also be seen as a physical interaction, we used a GST fusion of the full lumenal domain of CNX (GST-CNX_{K46-M417}), and the loop domain (GST-CNX_{M267-L412}), using GST as a control, and tested for ERp57 binding (FIG. 6A). The results show that ERp57 binds specifically to both GST-CNX fusions (FIG. 6B). Thus the functional³ and crosslinking² results showing the interaction of CNX and ERp57 were confirmed, and the regions that promote this interaction defined.

In alternative embodiments, protein regions homologous to yeast Ire1p may be used in systems of the invention. For example, a human Ire1p homologue has been characterized (see GenBank LOCUS AF059198, 3629 bp Homo sapiens protein kinase/endoribonulcease (IRE1) mRNA, complete mRNA, ACCESSION AF059198, VERSION AF059198.1 GI:3300093, dated 11 Jul. 1998; and, Tirasophon, et al., “A stress response pathway from the endoplasmic reticulum to the nucleus requires a novel bifunctional protein kinase/endoribonuclease (Ire1p) in mammalian cells” Genes Dev. 12 (12), 1812-1824 (1998)). Similarly, an Arabidopsis thaliana endoribonuclease/protein kinase Ire1p-like protein (IRE1b) has recently been described (see GenBank LOCUS AY057897, 2894 bp mRNA complete cds, ACCESSION AY057897, VERSION AY057897.1, GI:16506692, dated 27 Oct. 2001). The UPR in mammalian cells is thought to involve homologs of Ire1p; examples include the mouse ern1 and ern2 and human ERN1 and ERN2 gene products (Tirasophon, W., et al. (1998) A stress response pathway from the endoplasmic reticulum to the nucleus requires a novel bifunctional protein kinase/endoribonuclease (Ire1p) in mammalian cells. Genes Dev 12:1812-1824; Wang, X. Z., et al. (1998) Cloning of mammalian Ire1 reveals diversity in the ER stress responses. EMBO J. 17:5708-5717; Kaufman, R. J. (1999) Stress signaling from the lumen of the endoplasmic reticulum: coordination of gene transcriptional and translational controls. Genes Dev 13:1211-1233). The ribonuclease domain of Ire1p is similar to that of mammaliam RNase L (e.g human RnaseL, see Kaufman, R. J. (1999), Genes Dev 13:1211-1233), and in some embodiments homologues of RnaseL may be used as an ribonuclease domain in systems of the invention, for example in mammalian cells.

Various aspects of the present invention encompass nucleic acid or amino acid sequences that are homologous to other sequences, such as proteins or protein domains that are homologous to known Ire1p proteins or protein domains. As the term is used herein, an amino acid or nucleic acid sequence is “homologous” to another sequence if the two sequences have regions that are substantially identical and the functional activity of the sequences is conserved (for example, both sequences function as or encode a selected enzyme or promoter function; as used herein, the term ‘homologous’ does not infer evolutionary relatedness). Nucleic acid sequences may also be homologous if they encode substantially identical amino acid sequences, even if the nucleic acid sequences are not themselves substantially identical, a circumstance that may for example arise as a result of the degeneracy of the genetic code.

Two nucleic acid or protein sequences are considered substantially identical if, when optimally aligned, they share at least about 25% sequence identity in protein domains essential for conserved function. In alternative embodiments, sequence identity may for example be at least 50%, 70%, 75%, 90% or 95% in such regions, or throughout the sequences. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence alignment may also be carried out using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/. The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST programs may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (which may be changed in alternative embodiments to 1 or 0.1 or 0.01 or 0.001 or 0.0001; although E values much higher than 0.1 may not identify functionally similar sequences, it is useful to examine hits with lower significance, E values between 0.1 and 10, for short regions of similarity), M=5, N=4, for nucleic acids a comparison of both strands. For protein comparisons, BLASTP may be used with defaults as follows: G=11 (cost to open a gap); E=1 (cost to extend a gap); E=10 (expectation value, at this setting, 10 hits with scores equal to or better than the defined alignment score, S, are expected to occur by chance in a database of the same size as the one being searched; the E value can be increased or decreased to alter the stringency of the search.); and W=3 (word size, default is 11 for BLASTN, 3 for other blast programs). The BLOSUM matrix assigns a probability score for each position in an alignment that is based on the frequency with which that substitution is known to occur among consensus blocks within related proteins. The BLOSUM62 (gap existence cost=11; per residue gap cost=1; lambda ratio=0.85) substitution matrix is used by default in BLAST 2.0. A variety of other matrices may be used as alternatives to BLOSUM62, including: PAM30 (9,1,0.87); PAM70 (10,1,0.87) BLOSUM80 (10,1,0.87); BLOSUM62 (11,1,0.82) and BLOSUM45 (14,2,0.87). One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially identical is that the two sequences hybridize to each other under moderately stringent, or preferably stringent, conditions. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, stringent conditions are selected to be about 5° C. lower than the thermal melting point for the specific sequence at a defined ionic strength and pH.

In the context of the present invention, a moiety such as a nucleic acid or protein is “heterologous” if it is present by virtue of human intervention in a cell in which it is not naturally present, irrespective of whether the moiety is derived from the same species or a difference species. A cell into which has been introduced a foreign (heterologous) nucleic acid, is considered “transformed”, “transfected” or “transgenic” because it contains the heterologous nucleic acid introduced by human intervention. Progeny of the cell that is initially transformed with a recombinant nucleic acid construct is also considered “transformed”, “transfected” or “transgenic”. The invention provides vectors, such as vectors for transforming insect cells. The term “vector” in reference to nucleic acid molecule generally refers to a molecule that may be used to transfer a nucleic acid segment(s) from one cell to another. A recombinant nucleic acid is a nucleic acid molecule that has been recombined or altered by human intervention using techniques of molecular biology. Generally, a recombinant nucleic acid comprises nucleic acid segments from different sources ligated together, or a nucleic acid segment that is removed from the adjoining segments with which it is naturally joined.

In various aspects, the invention may utilize a variety of proteins or protein domains homologous to protein domains of Ire1p. It is well known in the art that some modifications and changes can be made in the structure of a polypeptide without substantially altering the biological function of that peptide, to obtain a biologically equivalent polypeptide. In one aspect of the invention, proteins used in the invention may differ from a portion of the corresponding native Ire1p or Ire1p homologue sequence by conservative amino acid substitutions. As used herein, the term “conserved amino acid substitutions” refers to the substitution of one amino acid for another at a given location in the peptide, where the substitution can be made without loss of function. In making such changes, substitutions of like amino acid residues can be made, for example, on the basis of relative similarity of side-chain substituents, for example, their size, charge, hydrophobicity, hydrophilicity, and the like, and such substitutions may be assayed for their effect on the function of the peptide by routine testing. In some embodiments, conserved amino acid substitutions may be made where an amino acid residue is substituted for another having a similar hydrophilicity value (e.g., within a value of plus or minus 2.0), where the following hydrophilicity values are assigned to amino acid residues (as detailed in U.S. Pat. No. 4,554,101, incorporated herein by reference): Arg (+3.0); Lys (+3.0); Asp (+3.0); Glu (+3.0); Ser (+0.3); Asn (+0.2); Gln (+0.2); Gly (0); Pro (−0.5); Thr (−0.4); Ala (−0.5); His (−0.5); Cys (−1.0); Met (−1.3); Val (−1.5); Leu (−1.8); Ile (−1.8); Tyr (−2.3); Phe (−2.5); and Trp (−3.4). In alternative embodiments, conserved amino acid substitutions may be made where an amino acid residue is substituted for another having a similar hydropathic index (e.g., within a value of plus or minus 2.0). In such embodiments, each amino acid residue may be assigned a hydropathic index on the basis of its hydrophobicity and charge characteristics, as follows: IlHe (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gln (−3.5); Asp (−3.5); Asn (−3.5); Lys (−3.9); and Arg (−4.5). In alternative embodiments, conserved amino acid substitutions may be made where an amino acid residue is substituted for another in the same class, where the amino acids are divided into non-polar, acidic, basic and neutral classes, as follows: non-polar: Ala, Val, Leu, Ile, Phe, Trp, Pro, Met; acidic: Asp, Glu; basic: Lys, Arg, His; neutral: Gly, Ser, Thr, Cys, Asn, Gin, Tyr.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the specification, the word “comprising” is used as an open-ended term, substantially equivalent to the phrase “including, but not limited to”, and the word “comprises” has a corresponding meaning. Citation of references herein shall not be construed as an admission that such references are prior art to the present invention. All publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

### REFERENCES

1. Zapun, A., Jakob, C. A., Thomas, D. Y., & Bergeron, J. J. Protein folding in a specialized compartment: the endoplasmic reticulum. Struct. Fold. Des. 7, 173-182 (1999).
2. Oliver, J. D., Roderick, H. L., Llewellyn, D. H. & High, S. ERp57 functions as a subunit of specific complexes formed with the ER lectins calreticulin and calnexin. Mol. Biol. Cell 10, 2573-2582 (1999).
3. Zapun, A. et al. Enhanced catalysis of ribonuclease B folding by the interaction of calnexin or calreticulin with ERp57. J. Biol. Chem. 273, 6009-6012 (1998).
4. Fields, S. & Song, O. A novel genetic system to detect protein-protein interactions. Nature 340, 245-246 (1989).
5. Gyuris, J., Golemis, E., Chertkov, H., & Brent, R. Cdi1, a human G1 and S phase protein phosphatase that associates with Cdk2. Cell 75, 791-803 (1993).
6. Johnsson, N. & Varshavsky, A. Ubiquitin-assisted dissection of protein transport across membranes. EMBO J. 13, 2686-2698 (1994).
7. Aronheim, A., Zandi, E., Hennemann, H., Elledge, S. J. & Karin, M. Isolation of an AP-1 repressor by a novel method for detecting protein-protein interactions. Mol. Cell. Biol. 17, 3094-3102 (1997).
8. Schwikowski, B., Uetz, P. & Fields, S. A network of protein-protein interactions in yeast. Nature Biotechnol. 18, 1257-1261 (2000).
9. Uetz, P. et al. A comprehensive analysis of protein-protein interactions in Saccharomyces cerevisiae. Nature 403, 623-627 (2000).
10. Walhout, A. J., Boulton, S. J. & Vidal, M. Yeast two-hybrid systems and protein interaction mapping projects for yeast and worm. Yeast 17, 88-94 (2000).
11. Walhout, A. J. et al. Protein interaction mapping in C. elegans using proteins involved in vulval development. Science 287, 116-122 (2000).
12. Stagljar, I., Korostensky, C., Johnsson, N. & te Heesen, S. A genetic system based on split-ubiquitin for the analysis of interactions between membrane proteins in vivo. Proc. Natl. Acad. Sci. U.S.A. 95, 5187-5192 (1998).
13. Broder, Y. C., Katz, S. & Aronheim, A. The Ras recruitment system, a novel approach to the study of protein-protein interactions. Curr. Biol. 8, 1121-1124 (1998).
14. Ehrhard, K. N., Jacoby, J. J., Fu, X. Y., Jahn, R. & Dohlman, H. G. Use of G-protein fusions to monitor integral membrane protein-protein interactions in yeast. Nature Biotechnol. 18, (2000).
15. Pelletier, M. F., Bergeron, J. J. M., &Thomas, D. Y. Molecular Chaperone Systems in the Endoplasmic Reticulum. In Molecular Chaperones: Frontiers in Molecular Biology Series. Peter Lund, ed. Oxford University Press. Oxford U.K. In press
16. Ellgaard, L., Molinari, M. & Helenius, A. Setting the standards: quality control in the secretory pathway. Science 286, 1882-1888 (1999).
17. Hubbard, S.C. & Robbins, P. W. Synthesis and processing of protein-linked oligosaccharides in vivo. J. Biol. Chem. 254, 4568-4576 (1979).
18. Zapun, A. et al. Conformation-independent binding of monoglucosylated ribonuclease B to calnexin. Cell 88, 29-38 (1997).
19. Rodan, A. R., Simons, J. F., Trombetta, E. S. & Helenius, A. N-linked oligosaccharides are necessary and sufficient for association of glycosylated forms of bovine RNase with calnexin and calreticulin. EMBO J. 15, 6921-6930 (1996).
20. Shamu, C. E. & Walter, P. Oligomerization and phosphorylation of the Ire1p kinase during intracellular signaling from the endoplasmic reticulum to the nucleus. EMBO J. 15, 3028-3039 (1996).
21. Chapman, R. E. & Walter, P. Translational attenuation mediated by a mRNA intron. Curr. Biol. 7, 850-859 (1997).
22. Travers, K. J. et al. Functional and genomic analyses reveal an essential coordination between the unfolded protein response and ER-associated degradation. Cell 101, 249-258 (2000).
23. Wang, X. Z. et al. Cloning of mammalian Ire1 reveals diversity in the ER stress responses. EMBO J. 17, 5708-5717 (1998).
24. Livnah, O. et al. An antagonist peptide-EPO receptor complex suggests that receptor dimerization is not sufficient for activation. Nature Struct. Biol. 5, 993-1004 (1998).
25. Remy, I., Wilson, I. A. & Michnick, S. W. Erythropoietin receptor activation by a ligand-induced conformation change. Science 283, 990-993 (1999).
26. Wilson, 1. A. & Jolliffe, L. K. The structure, organization, activation and plasticity of the erythropoietin receptor. Curr. Opin. Struct. Biol. 9, 696-704 (1999).
27. Liu, C. Y., Schroder, M. & Kaufman, R. J. Ligand-independent dimerization activates the stress response kinases IRE1 and PERK in the lumen of the endoplasmic reticulum. J. Biol. Chem. 275, 24881-24885 (2000).
28. Wada, I. et al. SSR alpha and associated calnexin are major calcium binding proteins of the endoplasmic reticulum membrane. J. Biol. Chem. 266, 19599-19610 (1991).
29. Ellgaard, L. et al. NMR structure of the calreticulin P-domain. Proc. Natl. Acad. Sci. U.S.A. 98, 3133-3138 (2001).
30. Ferrari, D. M. & Soling, H. D. The protein disulfide-isomerase family: unravelling a string of folds. Biochem J. 3391-10 (1999).
31. Shahinian, S. et al. Involvement of protein N-glycosyl chain glucosylation and processing in the biosynthesis of cell wall beta-1,6-glucan of Saccharomyces cerevisiae. Genetics 149, 843-856 (1998).
32. Guthrie, C. & Fink, G. R. Guide to yeast genetics and molecular biology. In Academic Press. San Diego (1991).
33. Mori, K. et al. A 22 bp cis-acting element is necessary and sufficient for the induction of the yeast KAR2 (BiP) gene by unfolded proteins. EMBO J. 11, 2583-2593 (1992).
34. Nantel, A., Mohammad-Ali, K., Sherk, J., Posner, B. I. & Thomas, D. Y. Interaction of the Grb10 adapter protein with the Raf1 and MEK1 kinases. J. Biol. Chem. 273, 10475-10484 (1998).
35. Zheng, C. F. & Guan, K. L. Properties of MEKs, the kinases that phosphorylate and activate the extracellular signal-regulated kinases. J. Biol. Chem. 268, 23933-23939 (1993).
36. Pelletier, M. F. et al. The heterodimeric structure of glucosidase II is required for its activity, solubility, and localization in vivo. Glycobiology 10, 815-827 (2000).

1. A cell for assaying interactions between fusion proteins, the cell comprising:
a) a first recombinant gene coding for a prey fusion protein, the prey fusion protein comprising:
i) a prey luminal domain having a first heterologous amino acid sequence;
ii) a prey kinase domain linked to the prey luminal domain;
iii) a prey transmembrane domain linking the prey luminal domain and the prey kinase domain, so that the prey fusion protein is anchored in a membrane with the prey luminal domain on the inside of the membrane and the prey kinase domain on the outside of the membrane;
b) a second recombinant gene coding for a bait fusion protein, the bait fusion protein comprising:
i) a bait luminal domain having a second heterologous amino acid sequence;
ii) a bait kinase domain linked to the bait luminal domain;
iii) a bait transmembrane domain linking the bait luminal domain and the bait kinase domain, so that the second fusion protein is anchored in the membrane with the bait luminal domain on the inside of the membrane and the bait kinase domain on the outside of the membrane;
c) wherein at least one of the bait and prey fusion proteins comprises an endoribonuclease domain linked to a kinase domain on the outside of the membrane and capable of eliciting a detectable signal in the cell when the endoribonuclease domain is activated; and,
d) wherein the bait and prey kinase domains are capable of auto-transphosphorylation upon dimerization of the first and second fusion proteins mediated by the first and second heterologous amino acid sequences, and transphosphorylation of the kinase domains activates the endoribonuclease domain to produce the detectable signal.

2. The cell of claim 1, wherein the rate of bait and prey kinase domain auto-transphosphorylation upon dimerization of the first and second fusion proteins is at least 5 times greater than the rate of bait kinase domain auto-phosphorylation or prey kinase domain auto-phosphorylation upon dimerization of either the bait fusion protein or prey fusion protein respectively.

3. The cell of claim 1, further comprising:
a) a prey signal sequence on the prey fusion protein targeting the prey fusion protein to the endoplasmic reticulum; and,
b) a bait signal sequence on the bait fusion protein targeting the bait fusion protein to the endoplasmic reticulum.

4. The cell of claim of any one of claim 3, wherein the cell is a yeast cell.

5. The cell of any claim 4, wherein the endoribonuclease domain is homologous to the Ire1p endoribonuclease domain.

6. The cell claim 5, wherein the bait kinase domain is homologous to the Ire1p kinase domain.

7. The cell of claim 6, wherein the prey kinase domain is homologous to the Ire1p kinase domain.

8. The cell of any one claim 7, wherein the bait transmembrane domain is homologous to the Ire1p transmembrane domain.

9. The cell of claim 8, wherein the prey transmembrane domain is homologous to the Ire1p transmembrane domain.

10. The cell of claim 1, wherein the dectectable signal is elicited when the endoribonuclease domain processes an mRNA.

11. The cell of claim 10, wherein the mRNA is homologous to HAC1 mRNA.

12. A system for assaying protein-protein interactions, comprising:
a) a prey fusion protein, the prey fusion protein comprising:
i) a prey luminal domain having a first heterologous amino acid sequence;
ii) a prey kinase domain linked to the prey luminal domain;
iii) a prey transmembrane domain linking the prey luminal domain and the prey kinase domain, so that the prey fusion protein is anchored in a membrane with the prey luminal domain on the inside of the membrane and the prey kinase domain on the outside of the membrane;
b) a bait fusion protein, the bait fusion protein comprising:
i) a bait luminal domain having a second heterologous amino acid sequence;
ii) a bait kinase domain linked to the bait luminal domain;
iii) a bait transmembrane domain linking the bait luminal domain and the bait kinase domain, so that the second fusion protein is anchored in the membrane with the bait luminal domain on the inside of the membrane and the bait kinase domain on the outside of the membrane;
c) wherein at least one of the bait and prey fusion proteins comprises an endoribonuclease domain linked to a kinase domain on the outside of the membrane and capable of eliciting a detectable signal when the endoribonuclease domain is activated; and,
d) wherein the bait and prey kinase domains are capable of auto-transphosphorylation upon dimerization of the first and second fusion proteins mediated by the first and second heterologous amino acid sequences, and transphosphorylation of the kinase domains activates the endoribonuclease domain to produce the detectable signal.

13. (Canceled)
